(19) United States
(12) Patent Application Publication
(10) Pub. No.: US 2016/0106390 A1
(43) Pub. Date: Apr. 21, 2016

(54) SYSTEMS AND METHODS FOR SECURING A PERIPHERAL ULTRASOUND DEVICE

(71) Applicant: GUARDSMAN SCIENTIFIC, INC., Park City, UT (US)

(72) Inventor: Daniel P. Vezina, Park City, UT (US)

(73) Assignee: Guardsman Scientific, Inc., Park City, UT (US)

(21) Appl. No.: 14/894,279

(22) PCT Filed: Jun. 9, 2014
(86) PCT No.: PCT/US2014/041593

§ 371 Date: Nov. 25, 2015

Related U.S. Application Data
(60) Provisional application No. 61/832,353, filed on Jun. 7, 2013.

(30) Foreign Application Priority Data
Jun. 7, 2013 (US) 61832353

(51) Int. Cl. A61B 8/00; A61B 8/06

(52) U.S. Cl. CPC A61B 8/4236; A61B 8/58; A61B 8/4444; A61B 8/06

(57) ABSTRACT

Implementations described and claimed herein provide systems and methods for securing a device for acquiring cardiac data points from a patient. In one implementation, the device includes a patient interface and a probe. The patient interface includes an anchor having a patient side adapted for attaching to a surface of the patient and a probe side disposed opposite the patient side. The patient interface further includes a synchronizer having a plurality of synchronization features corresponding to a plurality of imaging locations on the patient. The synchronizer is mounted to the anchor and adapted to engage the probe. A window extends through the anchor and the synchronizer. The plurality of synchronization features are positioned around the window. Each of the synchronization features includes a unique set of keys identifying one of the plurality of imaging locations.

### CROSS-REFERENCE TO RELATED APPLICATION

The present application claims benefit under 35 U.S.C. §119(e) to U.S. Provisional Patent Application No. 61/832,353, entitled “Systems and Methods Securing a Peripheral Ultrasound Device” and filed on Jun. 7, 2013. This application is specifically incorporated by reference herein in its entirety.

### TECHNICAL FIELD

Aspects of the present disclosure relate to systems and methods for acquiring circulatory system information from a patient and more particularly to the acquisition of cardiac data points reflecting the function of the heart. Some aspects of the present disclosure automatically and uninterruptedly acquire cardiac ultrasound-generated data points to optimize the hemodynamic management of a patient.

### BACKGROUND

Proper circulatory function is essential to sustain and prolong life. From a more practical standpoint, circulatory function can be a factor affecting healthcare costs resulting from complications, hospital readmissions, and mortality. According to some professionals, ensuring the adequacy of circulatory function is one of the most important clinical goals of healthcare providers when managing the well-being and clinical performance of patients. Many medical professionals endorse the use of the electrocardiogram (EKG) monitor, systemic blood pressure (BP), pulse oximetry (SpO2), and urine output (UO), known as conventional parameters, as the standard of care of assessing and managing a patient circulatory function.

Using the conventional parameters may be clinically acceptable for patients with normal cardiovascular function. However, the conventional parameters often provide incomplete information for patients with cardiovascular risk factors and/or comorbidities. For example, in various clinical settings, managing the circulatory function of a patient with diastolic dysfunction and or systolic dysfunction, also known as congestive heart failure (CHF) using only the conventional parameters and commonly used clinical strategies can lead a healthcare provider to deliver inappropriate pharmacologic and non-pharmacologic therapies, leading to volume overload of the circulatory system of the patient. As a result of the incomplete information, many patients are at risk of not receiving optimal hemodynamic management. This can lead to cardiovascular complications, major organ failure, hospital admission or readmission, and/or death. This result is both detrimental to the health of the patient and costly to the healthcare system.

The weaknesses in the current standard of care using the conventional parameters is compounded by the fact that CHF is the leading admission diagnosis for medicine and cardiology services in the United States. For example, diastolic dysfunction is often the underlying cause of CHF, and over 50% of individuals over 65 suffer from some degree of diastolic dysfunction, with 40% being mild cases and over 10% being moderate or severe. Further adding to the problem, diastolic dysfunction is common among the baby boomer population. The number of individuals over 65 has been projected to increase by 50% from 2000 to 2020, and as a result, the instances of CHF are likely to rise significantly.

It is with these observations in mind, among others, that various aspects of the present disclosure were conceived and developed.

### SUMMARY

Implementations described and claimed herein address the foregoing problems by providing systems and methods for securing a device for acquiring cardiac data points from a patient. In one implementation, the device includes a patient interface and a probe. The patient interface includes an anchor having a patient side adapted for attaching to a surface of the patient and a probe side disposed opposite the patient side. The patient interface further includes a synchronizer having a plurality of synchronization features corresponding to a plurality of imaging locations on the patient. The synchronizer is mounted to the anchor and adapted to engage the probe. A window extends through the anchor and the synchronizer. The plurality of synchronization features are positioned around the window. Each of the synchronization features includes a unique set of keys identifying one of the plurality of imaging locations.

In another implementation, a probe includes a housing having an interface side. A head is mounted within an opening in the interface side. The head configured to acquire data points from a patient. A synchronizer has a set of receptacles disposed in a groove extending around the opening. The set of receptacles is configured to receive a set of unique keys from a plurality of sets based on an orientation of the set of receptacles to a patient interface. Each of the plurality of sets corresponds to an imaging location on the patient.

In another implementation, a device includes an anchor having a side adapted for attaching to a surface of a target. A synchronizer is mounted to the anchor, and has a plurality of synchronization features corresponding to a plurality of imaging locations on the target. A window extends through the anchor and the synchronizer. The plurality of synchronization features are positioned around the window. Each of the synchronization features includes a unique set of keys identifying a particular imaging location from the plurality of imaging locations. A probe housing has an interface side, and a head is mounted within an opening in the interface side. The head is configured to acquire data points from the target along an imaging direction. A set of receptacles is disposed adjacent to the opening in the interface side. The set of receptacles is configured to match the unique set of keys in one of the synchronization features based on an orientation of the interface side to the window. A recognizer is configured to identify the particular imaging location based on the match between the unique set of keys and the set of receptacles.

In yet another implementation, a method securing a probe to a patient interface is provided. An anchor is attached to a surface of a patient at a particular imaging location. The anchor is mounted to a synchronizer having a plurality of synchronization features corresponding to a plurality of imaging locations on the patient. The plurality of synchronization features are positioned around a window extending through the anchor and the synchronizer. One of the synchronization features includes a unique set of keys identifying the particular imaging location from the plurality of imaging locations. A set of receptacles disposed adjacent to an opening in an interface side of a housing of the probe is aligned with the unique set of keys. A body extending from the opening in the interface side is coupled to the synchronizer based on the alignment of the receptacles with the unique set of keys. The coupling positions a head mounted within the opening in the interface side within the window. The head is configured to acquire data points from the patient along an imaging direction at the particular imaging location.

In still another implementation, one or more processor-readable storage media storing processor-executable instructions for performing a process are provided. The process identifies a match between a set of receptacles and a unique set of keys. The set of receptacles are disposed adjacent to an opening in an interface side of a probe housing, and the unique set of keys are disposed on one of a plurality of synchronization features positioned around a window in a patient interface. The plurality of synchronization features correspond to a plurality of imaging locations. The process determines a particular imaging location from the plurality of imaging locations based on the match between the set of receptacles and the unique set of keys.

Other implementations are also described and recited herein. Further, while multiple implementations are disclosed, still other implementations of the presently disclosed technology will become apparent to those skilled in the art from the following detailed description, which shows and describes illustrative implementations of the presently disclosed technology. As will be realized, the presently disclosed technology is capable of modifications in various aspects, all without departing from the spirit and scope of the presently disclosed technology. Accordingly, the drawings and detailed description are to be regarded as illustrative in nature and not limiting.

### BRIEF DESCRIPTION OF THE DRAWINGS

FIG. 1A shows a patient having a plurality of devices positioned in locations conducive to the acquisition of cardiac data points.

FIG. 1B illustrates an example system for the hemodynamic management of a patient.

FIG. 2 illustrates a bottom perspective view of an example patient interface.

FIG. 3 shows a top perspective view of the patient interface of FIG. 2.

FIG. 4 is a detailed view of a synchronizer of the patient interface of FIG. 2.

FIG. 5 is a side view of the patient interface of FIG. 2.

FIG. 6 illustrates a bottom perspective view and a detailed bottom perspective view of an example probe.

FIG. 7 shows a detailed bottom view of the probe of FIG. 6.

FIG. 8 is an exploded view of an example device for acquiring cardiac data points.

FIG. 9 shows a top view of one of the device of FIG. 8.

FIG. 10 is a bottom perspective view of the device of FIG. 8.

FIG. 11 is a detailed bottom perspective view of the device of FIG. 8.

FIG. 12 illustrates example operations for securing a device for acquiring cardiac data points from a patient.

FIG. 13A shows a kit including four patient interfaces and instructions.

FIG. 13B shows a kit including one patient interface and instructions.

FIG. 14 is an example computing system that may implement various systems and methods discussed herein.

### DETAILED DESCRIPTION

Aspects of the present disclosure provide patient hemodynamic management and associated systems and methodologies for acquiring cardiac data points from a patient. Aspects of the present disclosure further provide systems and methods for securing a device for acquiring cardiac data points, such as a peripheral ultrasound device, to a patient. In one aspect, the device includes a patient interface and a probe. In contrast to handheld devices, the patient interface securely positions the probe on the patient for hands-free capture of cardiac data points using one or more sensors, such as a transducer. The cardiac data points may include ultrasound-generated data points, particularly related to blood flow inside and in structures connected to the heart. It will be appreciated that the probe may include pressure, flow, impedance, conduction, electrical, and/or temperature sensors in lieu of or in addition to an ultrasonic transducer to capture various patient data points pertaining to the health status of the patient. The probe captures cardiac data points along an imaging direction. The transducer and/or other sensors may be automatically or manually adjusted to adjust the imaging direction for uninterrupted data acquisition. To facilitate efficient acquisition of cardiac data points, one or more patient interfaces may be placed at various imaging locations on the patient. When the probe engages a patient interface positioned on the patient, a synchronizer automatically identifies the imaging location. The cardiac data points captured at one or more imaging locations may be used to determine how the patient heart is functioning, determine clinical treatment strategies for the patient, and/or otherwise optimize the hemodynamic management of the patient.

The various systems and methods of the present disclosure provide for securing a probe or similar device on a patient and for automatically identifying the location of the probe on the patient. The example implementations discussed herein reference hemodynamic status or circulatory function as well as cardiac data points or ultrasound-generated data points. However, it will be appreciated by those skilled in the art that the presently disclosed technology is applicable to other patient conditions, statuses, and data as well as other sensor-generated data points. Furthermore, while the various systems and method are described herein with reference to human patients, it will be appreciated that the present disclosure applies to other animals or other animate or inanimate objects as well.

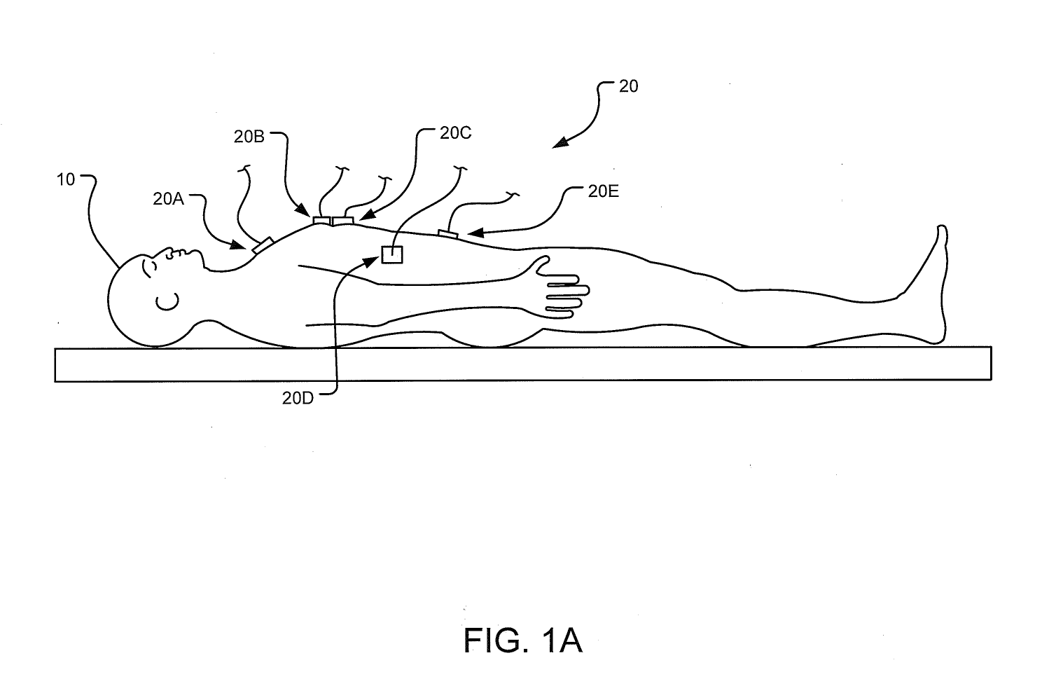

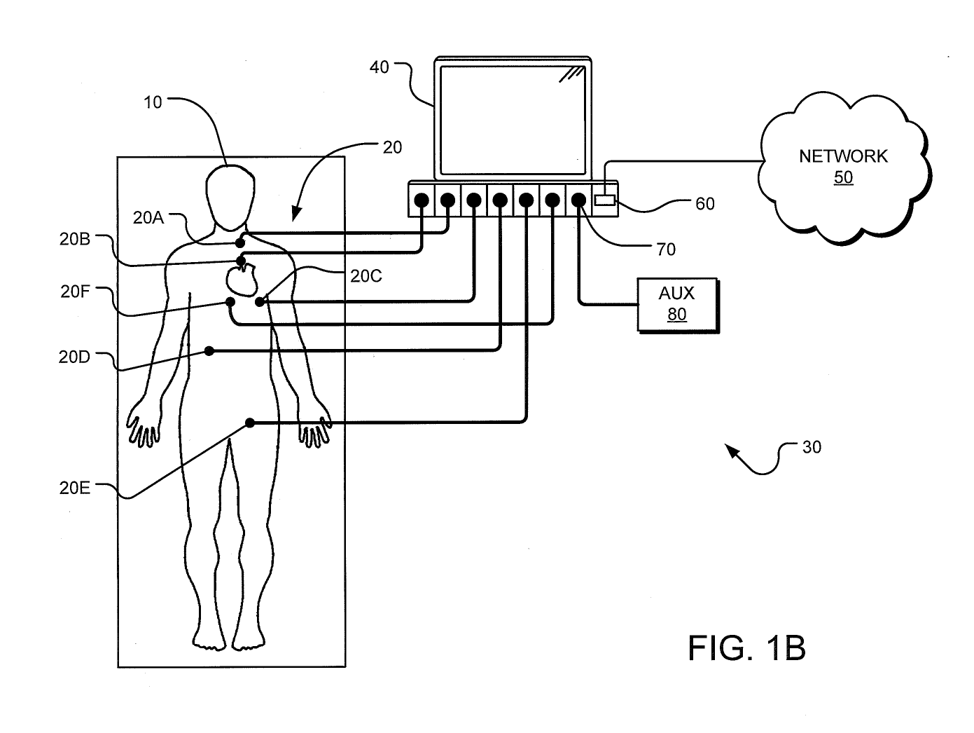

For a detailed description of an example system for the hemodynamic management of a patient, reference is made to FIGS. 1A-1B. In one implementation, one or more devices 20 are secured to a patient 10 to acquire cardiac data points or other patient information. Turning to FIG. 1A, the patient 10 is shown with the devices 20 positioned at different imaging locations generally on the anterior surface of the body of the patient 10. An imaging location, which may be internal or external to the body of the patient 10, is a location from which the heart may be imaged or from which cardiac or other patient data may be captured using the devices 20.

In the implementation shown in FIG. 1A, four devices 20A-20D are placed at a suprasternal notch imaging location, a transthoracic parasternal imaging location, a transthoracic apical imaging location, and a sub-costal imaging location, respectively, to capture cardiac data points. Additional devices 20 may be positioned at other imaging locations to capture additional cardiac data points and/or data points corresponding to more superficial structures, non-cardiac structures, and/or other anatomy or patient conditions depending on the needs of the patient. For example, an internal cardiac device 20F may be placed at a mid-esophageal imaging location, and an external non-cardiac device 20E may be positioned to capture non-cardiac structures outside the chest of the patient. In one implementation, each of the devices 20 are configured to identify the imaging location, as described herein.

As can be understood from FIG. 1B, in one implementation, a system 30 for hemodynamic management includes one or more devices 20 in communication with a user device 40, which may be used by a medical provider or other user to access and interact with acquired cardiac data points, hemodynamic management information, clinical strategies, or other patient information. The user device 40 may be any form of computing device, including, without limitation, a personal computer, a terminal, a workstation, a mobile phone, a mobile device, a tablet, a set top box, a multimedia console, a television, or the like. The various components of the system 30 may communicate in a variety of manners, for example, via a direct connection or indirectly via a network 50 (e.g., the Internet, an intranet, wired network, wireless network, etc.). In one implementation, the user device 40 includes a network interface 60 for facilitating communication between the user device 40 and various components of the system 30 via the network 50.

The network 50 may be used by one or more computing and data storage devices (e.g., one or more databases) for providing hemodynamic management of one or more patients. In one implementation, the network 50 includes a server hosting a website or an application that a user, such as a healthcare provider, the patient 10, or another authorized personnel, may visit to access the acquired cardiac data points or other information regarding the patient 10. The server may be a single server, a plurality of server with each server being a physical server or a virtual machine, or a collection of both physical servers and virtual machines. In another implementation, a cloud hosts one or more components of the system 30. The user device 40, the server, and other resources connected to the network 50 may access one or more other servers to access one or more websites, applications, web services interfaces, storage devices, computing devices, other network components, or the like. The serve may also host a search engine that the system 30 uses for accessing, search for, analyzing, modifying, or otherwise interacting with cardiac data points, clinical strategies, hemodynamic management data, and/or other stored data.

In one implementation, the devices 20 are in communication with the user device 40 to collect cardiac data points from the patient 10. The devices 20 may communicate with the user device 40 in a variety of manners, including, without limitation, a wired connection or a wireless connection (e.g., via the network 50). The devices 20 are each configured to alternate between sending and receiving signals. For example, the devices 20 may include ultrasonic transducers configured to intermittently or continuously produce and detect ultrasonic waves. However, one or more of the devices 20 may include pressure, flow, impedance, conduction, electrical, and/or temperature sensors in lieu of or in addition to an ultrasonic transducer to capture various patient data points pertaining to the health status of the patient 10. Furthermore, the user device 40 may include an auxiliary device interface 70 configured to communicate with one or more auxiliary devices 80 to obtain information relating to a hemodynamic or cardiovascular function status of the patient 10 or otherwise generally relating to the health or status of the patient 10. The auxiliary devices 80 may include, without limitation, an EKG, a blood pressure monitor, and the like. Based on the cardiac data points and other patient data points captured using the devices 20 and/or the auxiliary devices 80, the user device 40 or another component of the system 30 generates and optimizes clinical intelligence, for example, as described U.S. patent application Ser. No. 12/536,247, now U.S. Pat. No. 8,348,847, entitled “System and Method for Managing a Patient” and filed on Aug. 5, 2009; U.S. patent application Ser. No. 13/179,748, entitled “System and Method for Managing a Patient” and filed on Jul. 11, 2011; U.S. patent application Ser. No. 13/711,221, entitled “System and Method for Managing a Patient” and filed on Dec. 11, 2012; U.S. patent application Ser. No. 13/711,290, entitled “System and Method for Managing a Patient” and filed on Dec. 11, 2012; and U.S. patent application Ser. No. 13/912,763, entitled “System and Method for Analytics-Based Patient Management” and filed on Jun. 7, 2013, all of which are incorporated by reference in their entirety herein.

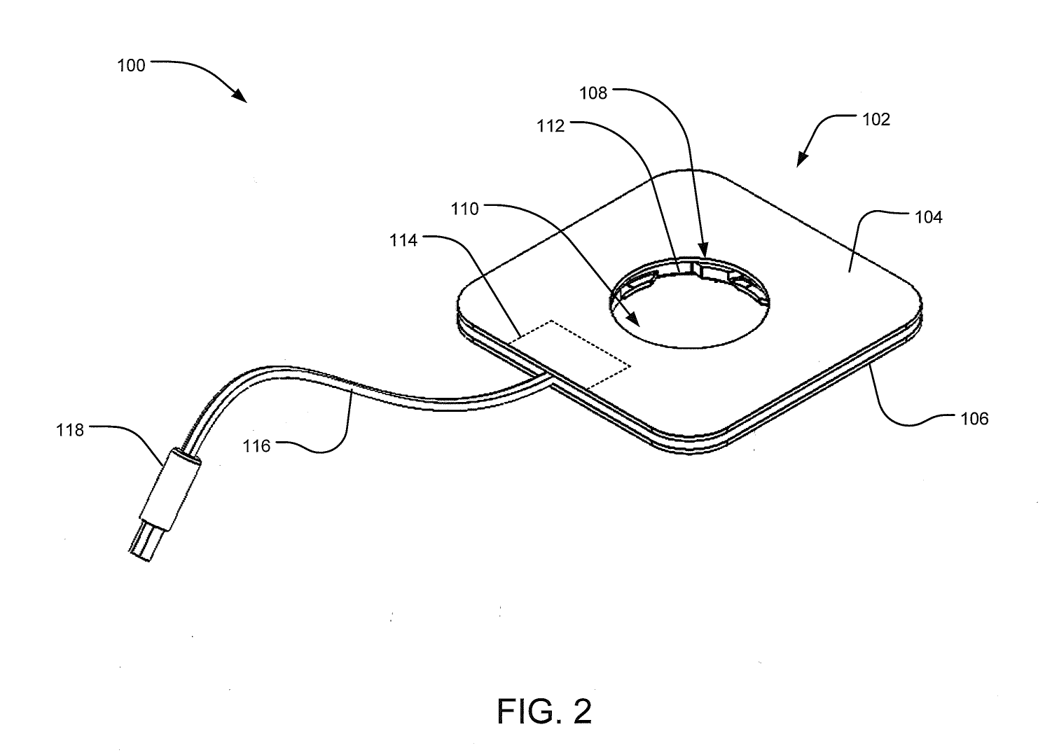

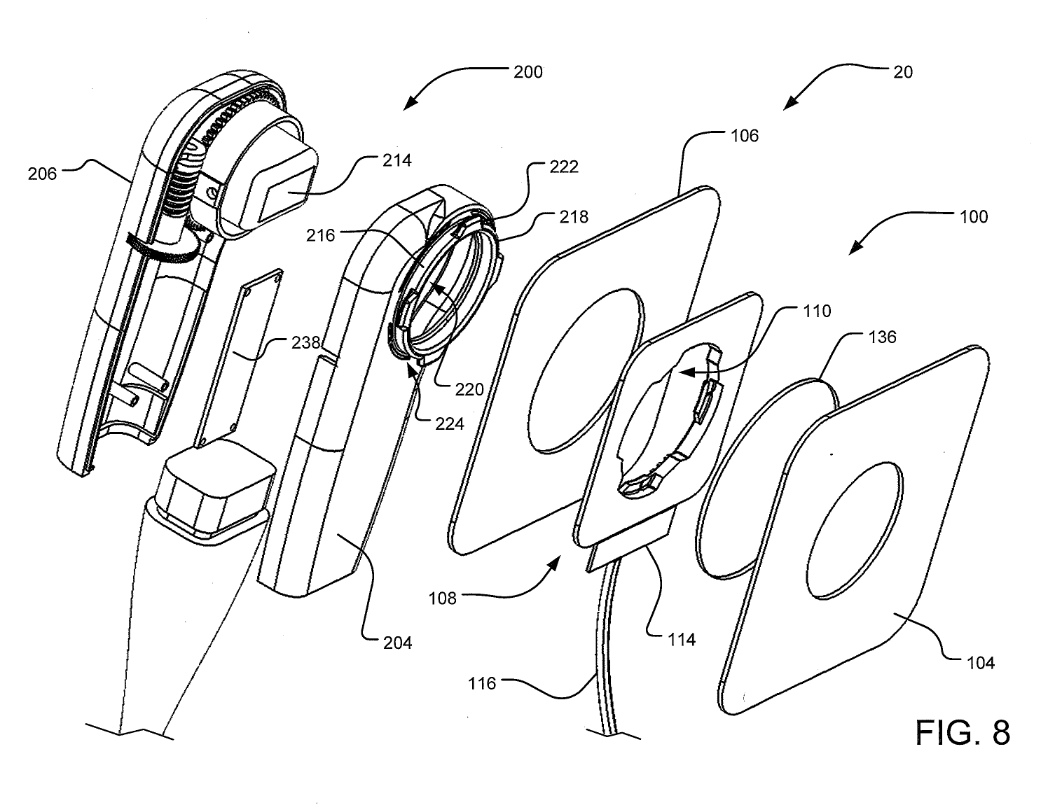

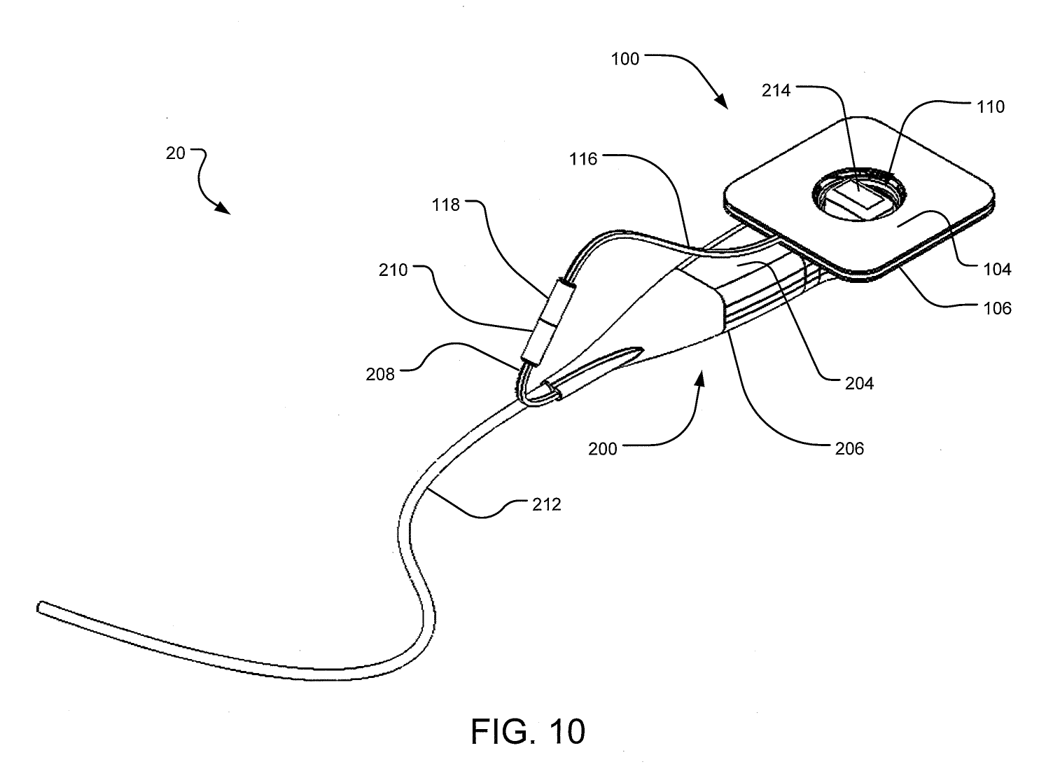

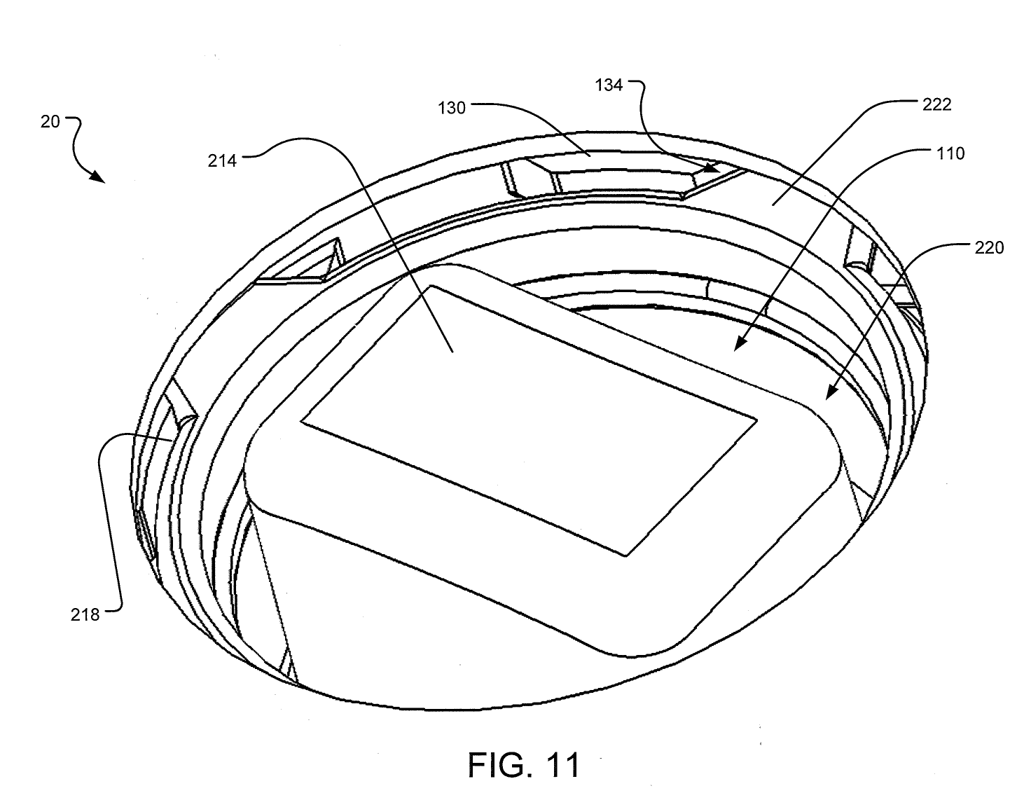

For a detailed description of systems and methods for securing the device 20 to the patient 10 for acquiring cardiac data points and/or other patient information, reference is made to FIGS. 2-12. In one implementation, the device 20 includes a patient interface 100 and a probe 200.

Turning to FIG. 2, a bottom perspective view of the patient interface 100 is shown. In one implementation, the patient interface 100 includes an anchor 102 having a patient side 104 configured to adhere or otherwise attach to an anterior surface of the body of the patient 10 and a probe side 106 positioned generally opposite the patient side 104. The anchor 102 may have one or more layers of material, including, without limitation, a soft, flexible biocompatible material that may conform to the contours of the body of the patient 10. In one implementation, the anchor 102 is a relatively thin patch approximately 4 inches wide and 4 inches long. However, other sizes and shapes, including, but not limited to, rectangular, circular, elliptical, triangular, angled, or contoured are contemplated. In one implementation, the sides 104 and 106 each are surfaces, which may be planar, contoured, textured, flexible, rigid, or the like, depending on the needs of the patient 10. The patient side 104 may include a membrane coated with an adhesive for attaching the patient interface 100 to the patient 10 at an imaging location. However, other mechanisms for attaching the patient interface 100 to the patient 10 are contemplated, including, without limitation, one or more straps, hooks, loops, elastics, hook and loop bands, belts, tie-downs, and/or the like, for example, attached to edges of the anchor 102 and wrapped around the body of the patient 10.

In one implementation, the patient interface 100 includes a synchronizer 108 configured to engage and synchronize with the probe 200. The synchronizer 108 and the anchor 102 may each have openings that generally align to form a window 110 sized and shaped to accommodate at least a portion of the probe 200 for acquiring data from the patient 10 when the synchronizer 108 is engaged to the probe 200. In one implementation, the synchronizer 108 includes a rim 112 extending around the window 110.

The synchronizer 108 may include various features for engaging and securing the probe 200. For example, the synchronizer 108 may include one or more mechanical features sized, shaped, and otherwise adapted to engage corresponding features on the probe 200. However, it will be appreciated that the synchronizer 108 may include other mechanical, magnetic, electrical, and/or optical features for engaging the probe 200. In one implementation, engaging the synchronizer 108 to the probe 200 establishes communication between the synchronizer 108 and the probe 200. Stated differently, the synchronizer 108 includes a recognizer 114 configured to initiate or otherwise establish communication with the probe 200 upon a coupling of the patient interface 100 with the probe 200. The recognizer 114 may include one or more processors or integrated circuits configured to execute various operations for identifying, authorizing, calibrating, and/or otherwise using the probe 200.

In one implementation, once communication is established between the recognizer 114 and the probe 200, the recognizer 114 identifies and calibrates the probe 200. The recognizer 114 detects the presence of the probe 200 and determines whether the probe 200 is authorized to operate with the patient interface 100. Stated differently, the recognizer 114 protects against the unauthorized or inadvertent use of the probe 200 and confirms that the probe 200 is capable of operating properly with the patient interface 100. In one implementation, the recognizer 114 authorizes the use of the probe 200 by determining whether the patient interface 100 synchronizes with the probe 200. The synchronizer 108 of the patient interface 100 may synchronize with the probe 200 using any form of unique communication, including, without limitation, a mechanical, electrical, optical, magnetic, radio frequency, wireless, and/or other communication format. For example, the synchronizer 108 may include: a set of keys that uniquely match a set of receptacles of the probe 200 to synchronize the patient interface 100 with the probe 200; a unique bar code that the probe 200 is configured to read to synchronize the patient interface 100 with the probe 200; a circuit that is open when the probe 200 is not connected to the synchronizer 108 and closed when the probe 200 is connected to the synchronizer 108; or the like. The synchronizer 108 may include a cable 116 with a connector 118 for connecting to the probe 200, the user device 40, a power source, or other resource for power and to facilitate communication. However, other power sources, including batteries, are contemplated, and the synchronizer 108 may include one or more interfaces for wireless, radio frequency, Bluetooth, or similar communication.

Once the recognizer 114 identifies and authorizes the use of the probe 200, in one implementation, the probe 200 is calibrated. The probe 200 may be calibrated by the recognizer 114, the user device 40, and/or one or more components of the probe 200. The calibration confirms the various components of the probe 200 are functioning properly, the probe 200 is properly coupled to the patient interface 100, and/or that the probe 200 is ready for use to capture patient data points. In one implementation, the recognizer 114, the user device 40, and/or one or more components of the probe 200 determines an imaging location of the device 20 on the patient 10 based on the synchronization of the patient interface 100 with the probe 200. The probe 200 may be oriented based on the determined imaging location to facilitate efficient acquisition of patient data points.

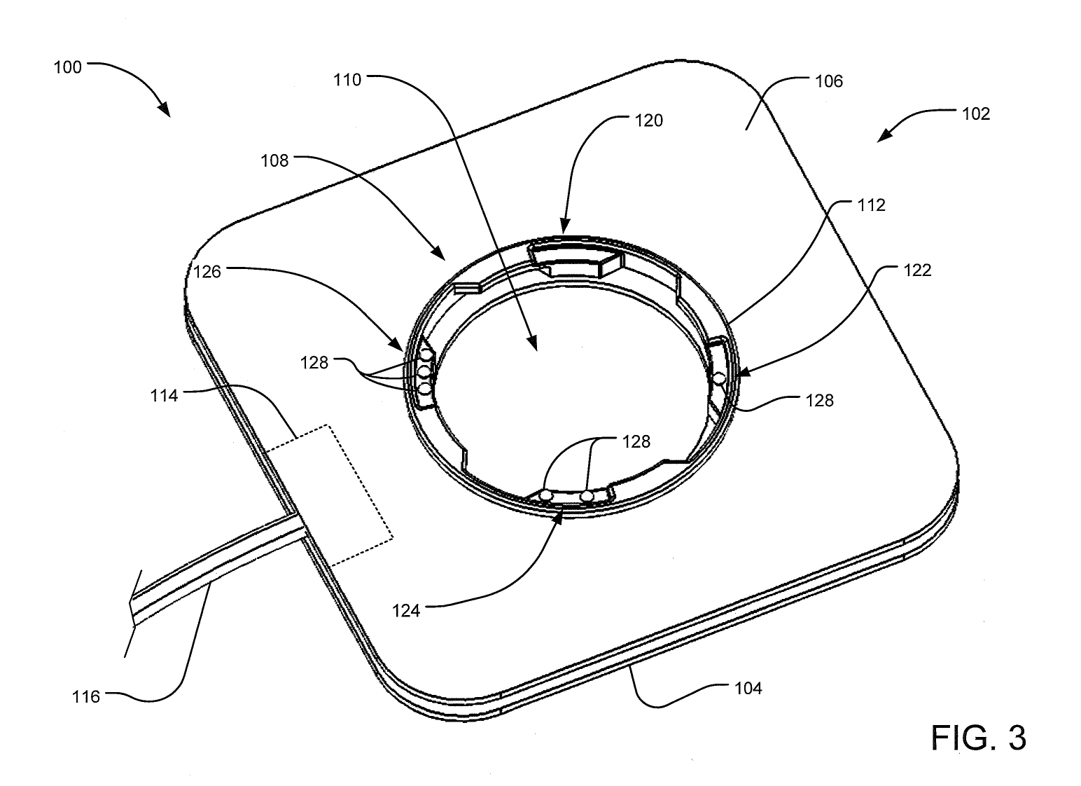

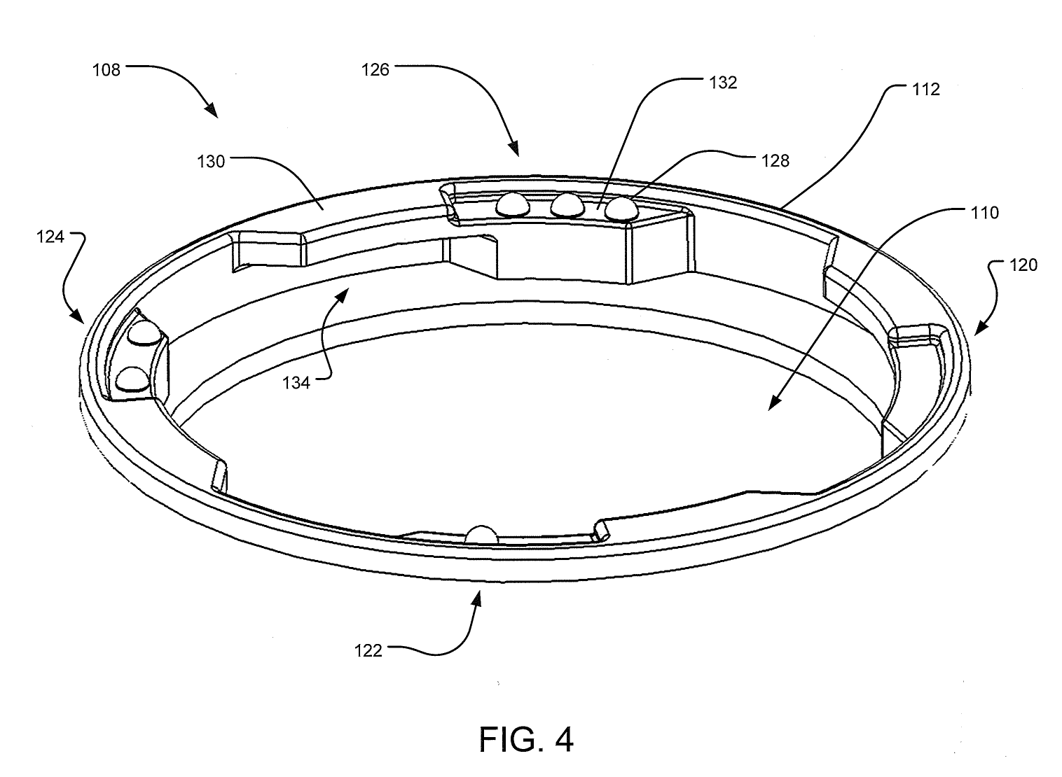

For a detailed description of the synchronization of the patient interface 100 with the probe 200, reference is first made to FIGS. 3-4, which show a top perspective view of the patient interface 100 and a detailed view of the synchronizer 108, respectively.

In one implementation, the synchronizer 108 includes one or more synchronization features disposed at various positions. In the example implementation shown in FIGS. 3-4, synchronization features are disposed at a first position 120, a second position 122, a third position 124, and a fourth position 126 around the window 110. The positions 120-126 may be spaced evenly around the window 110, for example, with each position disposed at a right angle relative to an adjacent position. The positions 120-126 may each correspond to a particular imaging location. For example, in the context of hemodynamic management, the positions 120-126 may correspond to a generic imaging location, a parasternal imaging location, an apical imaging location, and a subcostal imaging location, respectively.

Each of the synchronization features at the positions 120-126 is unique and distinct from each other and configured to communicate with the probe 200. In one implementation, the synchronizer 108 may include a set of keys 128 disposed relative to the positions 120-126 to create unique and distinct synchronization features. The positions 120-126 may be unique and distinct from each other based on the number of keys 128 at each position, the shape of the keys 128 at each position, the size of the keys 128 at each position, the format of the keys 128 at each position (e.g., mechanical, electrical, magnetic, optical, etc.), and/or the like. For example, as shown in FIGS. 3-4, the first position 120 includes no keys 128, the second position 122 includes one key 128, the third position 124 includes two keys 128, and the fourth position 126 includes three keys 128. Each of the keys 128 shown in FIGS. 3-4 is a contoured protrusion. However, it will be appreciated that the keys 128 may be a variety of shapes forming a male or female component, including without limitation, a protrusion that is cylindrical, conical, pyramidal, spherical, cubical, angular, contoured, or the like or an indent that is similarly shaped. Further, the keys 128 may be replaced or supplemented with other mechanical, electrical, optical, and/or magnetic features.

As can be understood from FIG. 4, in one implementation, the synchronization features each include a projection 130 extending from the rim 112 of the synchronizer 108 towards a general center of the window 112. The projection 130 includes a shelf 132 and a channel 134 defined therein. In one implementation, the shelf 132 is formed by an indent in the projection 130, such that the shelf 132 is an open surface facing in the direction of the probe side 106. Any keys 128 may be positioned on and extending away from the shelf 132 to facilitate synchronization with corresponding features of the probe 200. In one implementation, the channel 134 connects to an opening adjacent to the projection 130 to form a female mechanical connection for engaging the probe 200, for example, using an insert and twist coupling.

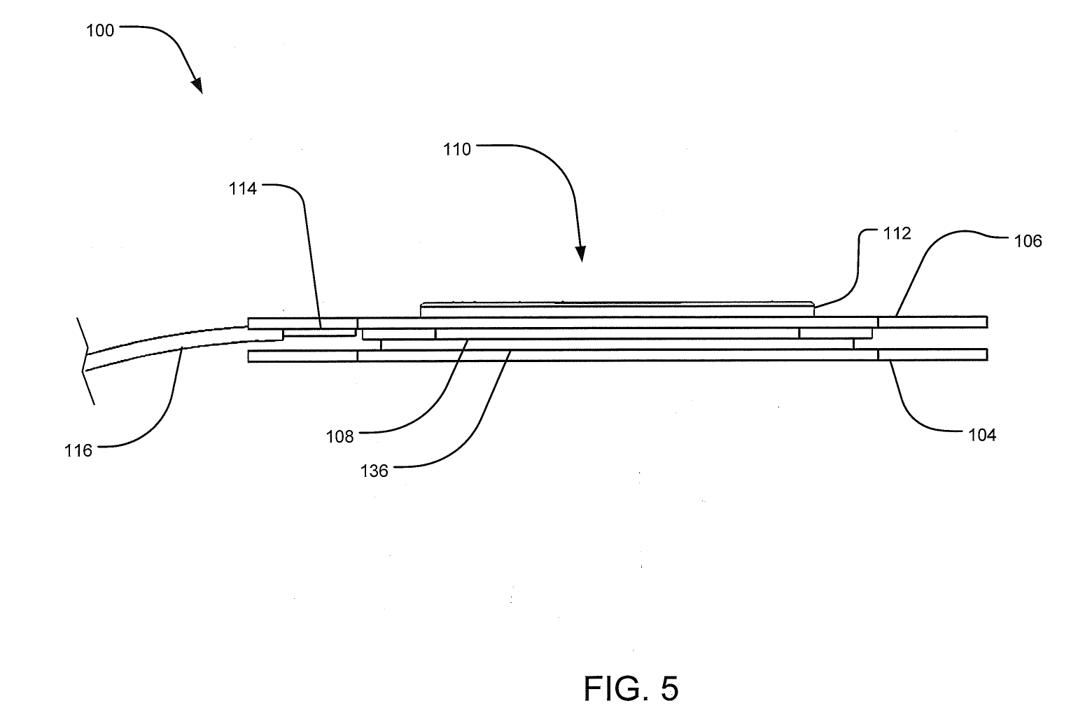

As described herein, coupling the synchronizer 108 to the probe 200 using, for example, the projection 130 and the channel 134, synchronizes the set of keys 128 to uniquely matching receptacles in the probe 200, and positions the probe 200 relative to the window 110 to facilitate capture of patient data points through the window 110. As can be understood from FIG. 5, in one implementation, a pad 136, such as an ultrasonic solid gel pad or liquid may occupy at least a portion of the window 110. The pad 136 is configured to facilitate continual contact between the probe 200 and the skin of the patient 10. In a specific implementation, the pad 136 may comprise a material conducive to transmitting ultrasonic signals. The pad 136 may be a material having a density similar to the body of the patient 10.

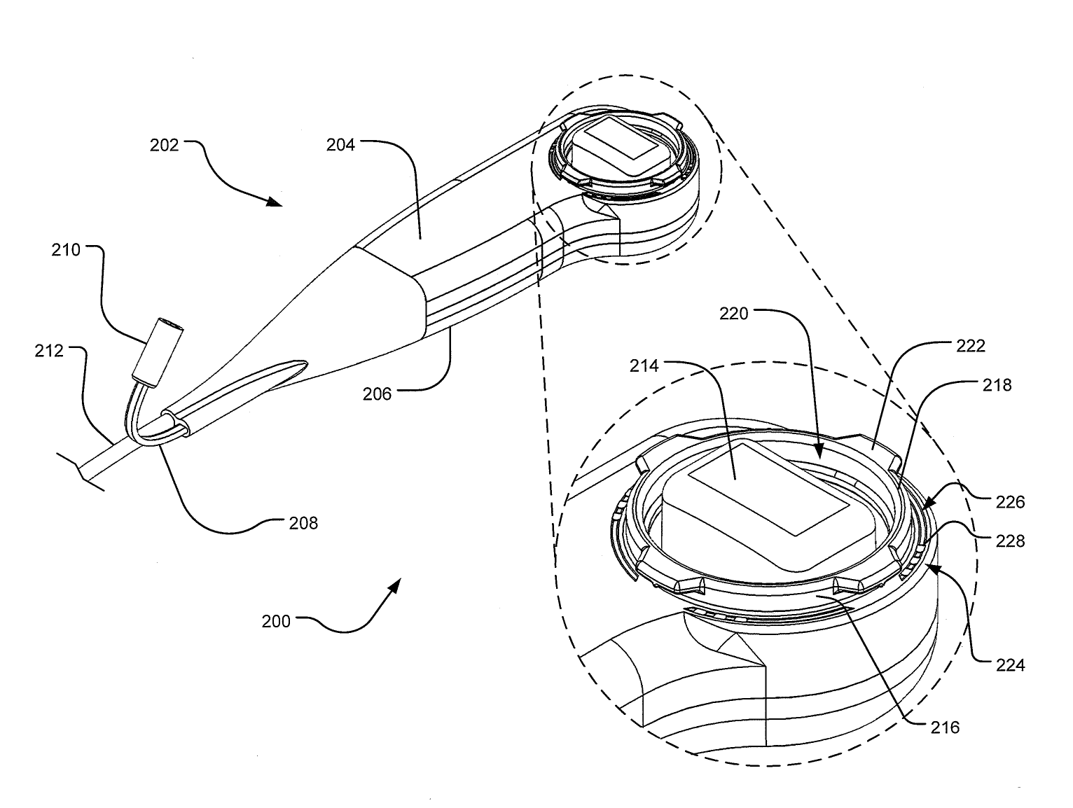

For a detailed description of the probe 200, reference is made to FIGS. 6-8. In one implementation, the probe 200 includes a housing 202 having an interface side 204 and a user side 206 positioned generally opposite the interface side 204. The housing 202 may include one or more cables extending therefrom for connecting to the patient interface 100, the user device 40, a power source, or other resource for power and to facilitate communication with the various components of the system 30. For example, a cable 208 may have a connector 210 configured to engage the connection 118 of the patient interface 100 and/or a cable 212 for connecting to the user device 40. However, other power sources, including batteries, are contemplated, and the probe 200 may include one or more interfaces for wireless, radio frequency, Bluetooth, or similar communication.

In one implementation, the probe 200 includes a head 214 having a transducer, imager, or similar acquisition mechanism configured for sending and receiving signals, including patient data points. For example, the head 214 may include an ultrasound transducer, or other imagers, including, without limitation, an x-ray imager, a computed tomography (CT) imager, a magnetic resonance imager (MRI), or the like. The head 214 can have a signal emitting surface adapted for interaction with the patient 10. As such, the signal emitting surface of the head 214 may be generally flat or contoured to suitably engage the surface of the patient 10. The head 214 may be a variety of shapes, including, but not limited to rectangular, circular, angled, contoured, or the like.

In one implementation, the head 214 is mounted within an opening 220 defined by an edge 218 of a body 216 such that it can rotate about an axis generally orthogonal to the interface side 204 of the housing 202. Additionally or alternatively, the head 214 can be mounted within the opening 220 such that it can pivot about an axis generally parallel to the interface side 204. It will be appreciated that the head 214 may be mounted in the opening 220 in a variety of manners. In some implementations, the head 214 is mounted directly to the housing 202 where the mounting allows for rotation and/or pivoting of the head 214 to adjust the orientation and/or direction of the imaging direction of the head 214, along which patient data is acquired. The head 214 may be positioned relative to the housing 202 in a position to interact with the surface of the patient 10. As such, in some implementations, the head 214 extends beyond the interface side 204 an amount approximately equal to the thickness of the patient interface 100, such that the head 214 extends into the window 110 through which signals (e.g., ultrasonic signals) may be directed during acquisition when the probe 200 is engaged to the patient interface 100. In other implementations, the head 214 is mounted more flush with the interface side 204 or even recessed relative thereto.

In one implementation, the head 214 is mounted using an adjuster, which is configured to adjust the orientation and/or direction of the head 214 to adjust the imaging direction along which patient data is acquired. For a detailed discussion of the systems and methods for adjusting the head 214, reference is made to U.S. patent application Ser. No. 12/646,617, entitled “Peripheral Ultrasound Device” and filed on Dec. 23, 2009, which is incorporated herein in its entirety. The adjustment of the head 214, data acquisition, and other operations of the probe 200 may be controlled by a controller 238 disposed in the housing 202. The controller 238 may include one or more processors or integrated circuits configured to execute various operations for controlling the probe 200, as well as interfacing, controlling, or otherwise communicating with the patient interface 100, the user device 40, and/or other components of the system 30.

The body 216 is configured to engage the synchronizer 108 to position the head 214 in the window 110 of the patient interface 100. In one implementation, a size and shape of the edge 218 of the body 216 mirrors the size and shape of the rim 112 of the synchronizer 108. The body 216 includes one or more wings 222 extending from the edge 218 configured to be received in the channel 134 of the synchronizer 108 via a twisting motion to couple the probe 200 to the patient interface 100. Stated differently, the edge 218 and wings 222 form a male mechanical connector and rim 112 with the projections 130 and channels 134 form a corresponding female mechanical connector. The male-female mechanical connectors form an insert and twist connection providing serial coupling and uncoupling of the probe 200 from the patient interface 100. In another implementation, the body 216 of the probe 200 and the synchronizer 108 of the patient interface 100 include mating press-fit or snap on mechanical connectors. Other connectors and coupling mechanisms are contemplated.

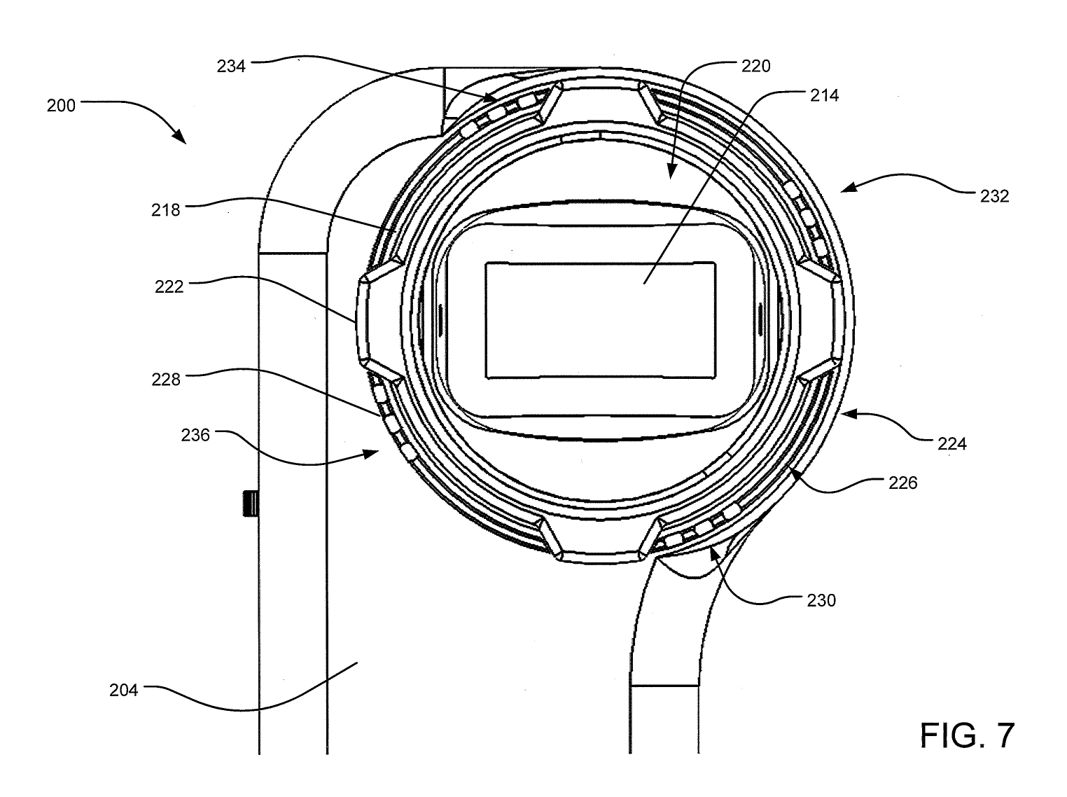

As can be understood from FIGS. 6-7, the probe 200 includes a corresponding synchronizer 224 for synchronizing with the synchronizer 108 of the patient interface 108. In one implementation, the corresponding synchronizer 224 includes a groove 226 with a set of receptacles 228. The groove 226 is shaped to align with the set of keys 128. Stated differently, the groove 226 is adapted to direct the keys 128 to the receptacles 228 as the synchronizer 108 is moved to engage the body 216.

In one implementation, the receptacles 228 are disposed at various locations along the groove 226 to form one or more groupings. In the example implementation shown in FIG. 7, the set of receptacles 228 are collected in a first grouping 230, a second grouping 232, a third grouping 234, and a fourth grouping 236 along the groove 226. The groupings 230-236 may be spaced evenly around the groove 226, for example, with each grouping positioned at a right angle relative to an adjacent grouping. The set of receptacles 228 are adapted to uniquely match with the set of keys 118 to synchronize the patient interface 100 with the probe 200. Each of the receptacles shown in the example of FIGS. 6-7 are contoured indents configured to matingly receive a key 118. However, it will be appreciated that the receptacles 228 may be a variety of shapes forming a male or female component, including without limitation, a protrusion that is cylindrical, conical, pyramidal, spherical, cubical, angular, contoured, or the like or an indent that is similarly shaped. Further, the receptacles 228 may be replaced or supplemented with other mechanical, electrical, optical, and/or magnetic features.

The number of receptacles 228 in each of the groupings 230-236 depends on the maximum number of keys 118 included at any of the positions 120-126. For example, the maximum number of keys 118 in the implementation shown in the Figures is three because the fourth position 126 of the synchronizer 108 includes three keys 128, which is more than any other the other positions 120-126. Accordingly, each of the groupings 230-236 includes three receptacles 228. Having the number of receptacles 228 in each of the groupings 230-236 equal to the maximum number of keys 118 in any of the positions 120-126 ensures that the patient interface 100 can synchronize with the probe 200 in various orientations. In other words, for example, because each of the groupings 230-236 includes three receptacles 228, any of the groupings 230-236 can receive the keys 118 from any of the positions 120-126. The orientations available for synchronization correspond to imaging locations.

In one implementation, the available orientations of the probe 200 depends on an alignment of the groupings 130-136 relative to the positions 120-126. In the implementation shown in the Figures, four different orientations are available based on which of the groupings 130-136 aligns with and receives which set of keys 118 in the positions 120-126. For example, a first orientation is defined by the receptacles 228 in the third grouping 234 receiving the set of keys 118 in the second position 122; a second orientation is defined by the receptacles 228 in the third grouping 234 receiving the set of keys 118 in the third position 124; a third orientation is defined by the receptacles 228 in the third grouping 234 receiving the set of keys 118 in the fourth position 126; and a fourth orientation is defined by the receptacles 228 in the third grouping 234 receiving the set of keys 118 in the first position 120. Stated differently, the orientation may be defined by which of the positions 120-126 is received by the third grouping 234, and because each of the positions 120-126 corresponds to an imaging location, the imaging location may be determined based on which of the positions 120-126 is received by the third grouping 234. Accordingly, in the example provided above, because the positions 120-126 correspond to a generic imaging location, a parasternal imaging location, an apical imaging location, and a subcostal imaging location, respectively, the first orientation defines the parasternal imaging location, the second orientation defines the apical imaging location, the third orientation defines the subcostal imaging location, and the fourth orientation defines the generic imaging location.

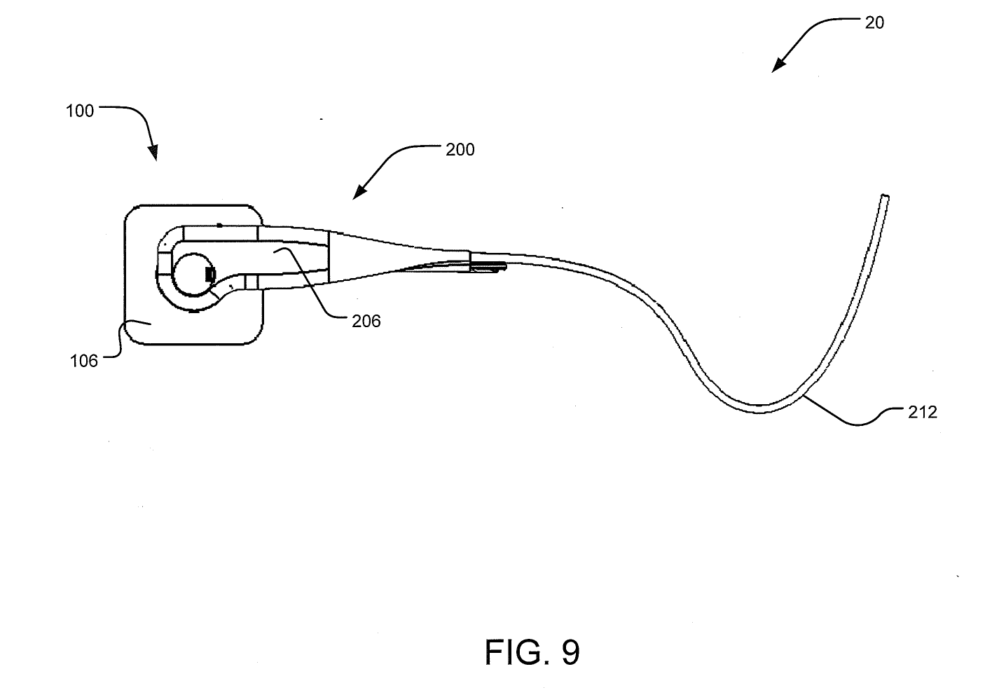

In one implementation, the third grouping 234 is positioned near a visual cue or positioned at a top end of the groove 226 to assist the user in properly synchronizing the probe 200 with the patient interface 100 to identify the imaging location. A user, such as a healthcare provider or other authorized personnel position one or more of the patient interfaces 100 at various imaging locations on the body of the patient 10. The user then aligns the third grouping 234, for example, using the visual cue, with the key position (e.g., 120, 122, 124, or 126) corresponding to the imaging location at which the patient interface 100 is positioned. Once the desired set of keys 118 is aligned with the third grouping 234, the body 216 of the probe 200 is engaged to the synchronizer 108 of the patient interface 100, as shown in FIGS. 9-11, aligning the head 214 of the probe 200 with the window 110 of the patient interface 100. The set of keys 118 at each of the positions 120-126 synchronizes with the corresponding grouping 230-236 of receptacles 228. Based on the synchronization, the recognizer 114, the controller 238, and/or the user device 40 determines the imaging location and/or calibrates the probe 200.

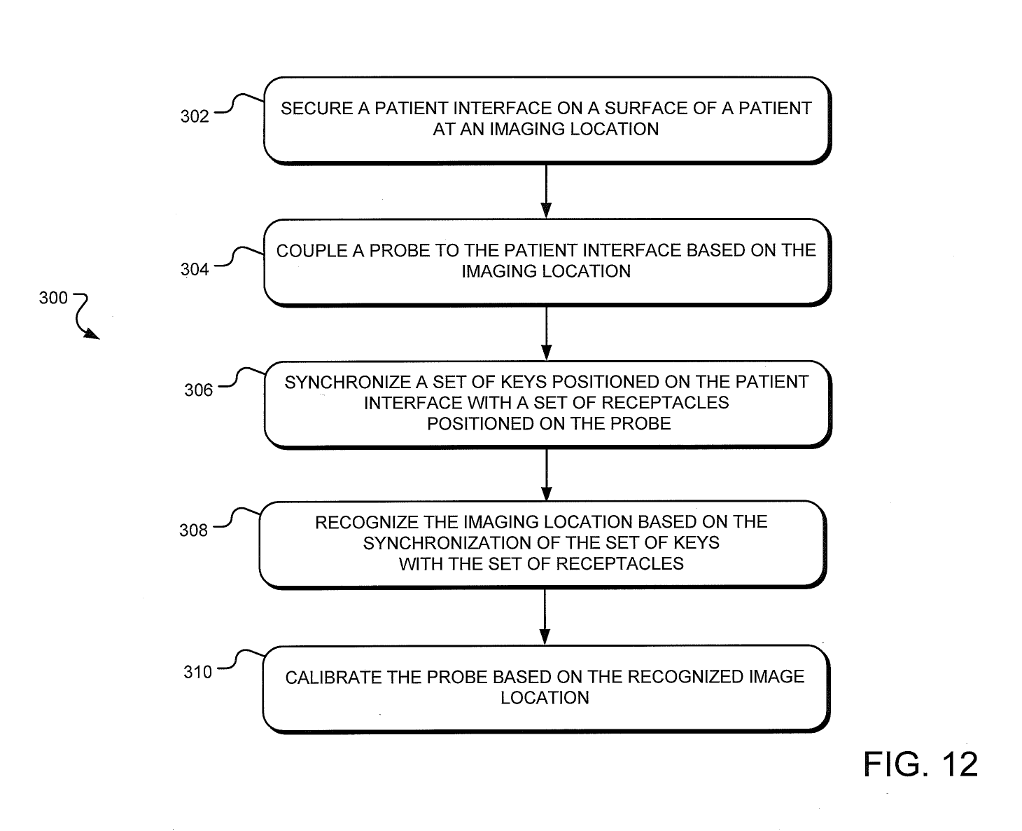

FIG. 12 illustrates example operations 300 for securing a device for acquiring cardiac data points from a patient. In one implementation, a positioning operation 302 positions at least one patient interface on a surface of the patient at an imaging location. The imaging location may be, for example, parasternal, apical, subcostal, suprasternal, and generic. The positioning operation 302 may utilize an adhesive surface on an anchor to secure the patient interface to the surface of the patient.

A coupling operation 304 couples a probe to the patient interface by engaging a synchronizer of the patient interface with a body extending from the probe. In one implementation, the synchronizer includes one or more channels, each configured to engage a wing of the probe. The coupling operation 304 couples the probe to the patient interface based on the imaging location. Stated differently, the probe may be oriented at various positions relative to the patient and the patient interface depending on the imaging location.

A synchronizing operation 306 synchronizes the patient interface with the probe. In one implementation, the synchronizing operation 306 synchronizes a set of keys positioned on the patient interface with a set of receptacles positioned on the probe. The set of keys and the set of receptacles may be corresponding mechanical, electrical, optical, magnetic, or other features. In one implementation, the set of keys and the set of receptacles are adapted to couple and communicate using a unique format.

A recognizing operation 308 identifies the image location based on the synchronization of the patient interface with the probe. In one implementation, the recognition operation 308 identifies the imaging location based on the manner in which the set of keys and the set of receptacles mechanically or otherwise couple or align. For example, the recognition operation 308 may identify the imaging location based on one of four orientations of the set of keys relative to the set of receptacles. Position 1, defined as having a top grouping of receptacles on the probe coupled with a set of keys having one key, indicates the parasternal imaging location. Position 2, defined as having the top grouping of receptacles on the probe coupled with a set of keys having two keys, indicates the apical imaging location. Position 3, defined as having the top grouping of receptacles on the probe coupled with a set of keys having three keys, indicates a subcostal imaging location. Position 4, defined as having the top grouping of receptacles on the probe coupled with a set of keys having no keys, indicates a generic window that can be defined by a user.

In one implementation, a calibrating operation 310 calibrates the probe based on the imaging location and the synchronization of the patient interface and the probe. The calibration operation 310 may configure the probe to operate based on the imaging location. Further, the calibration operation 310 may ensure that the probe is permitted to operate and capable of operating properly with the patient interface. The calibration operation 310 may additionally initiate a predetermined calibration and safety check process of the electrical components of the probe before it can be used on the patient.

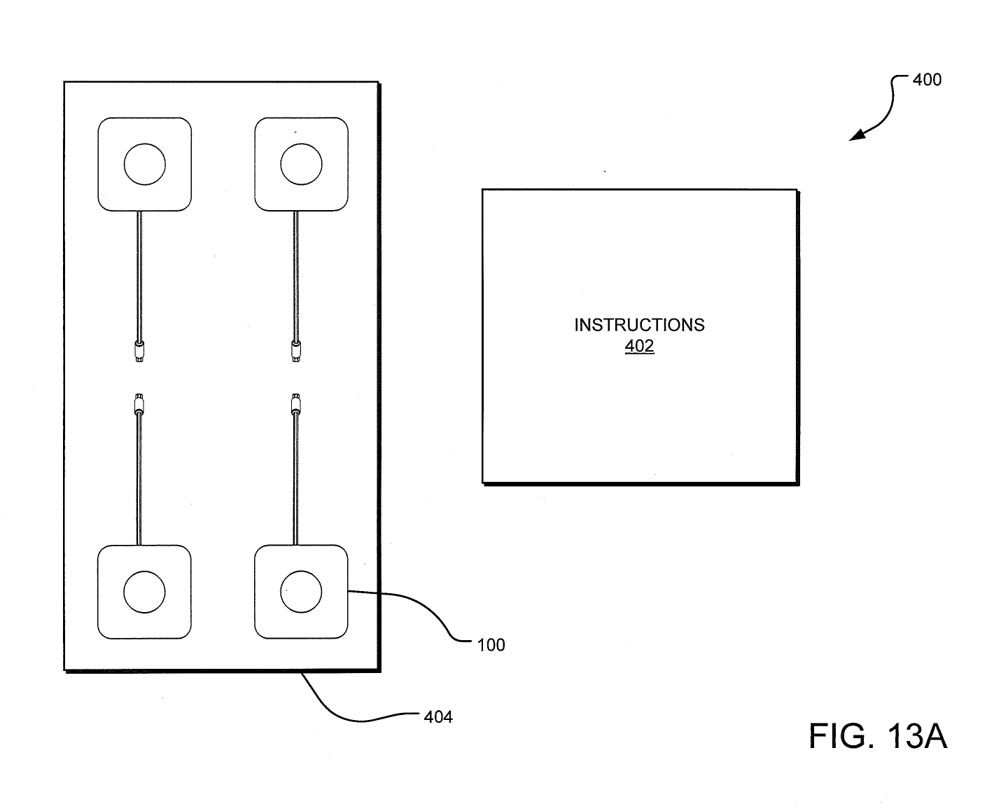

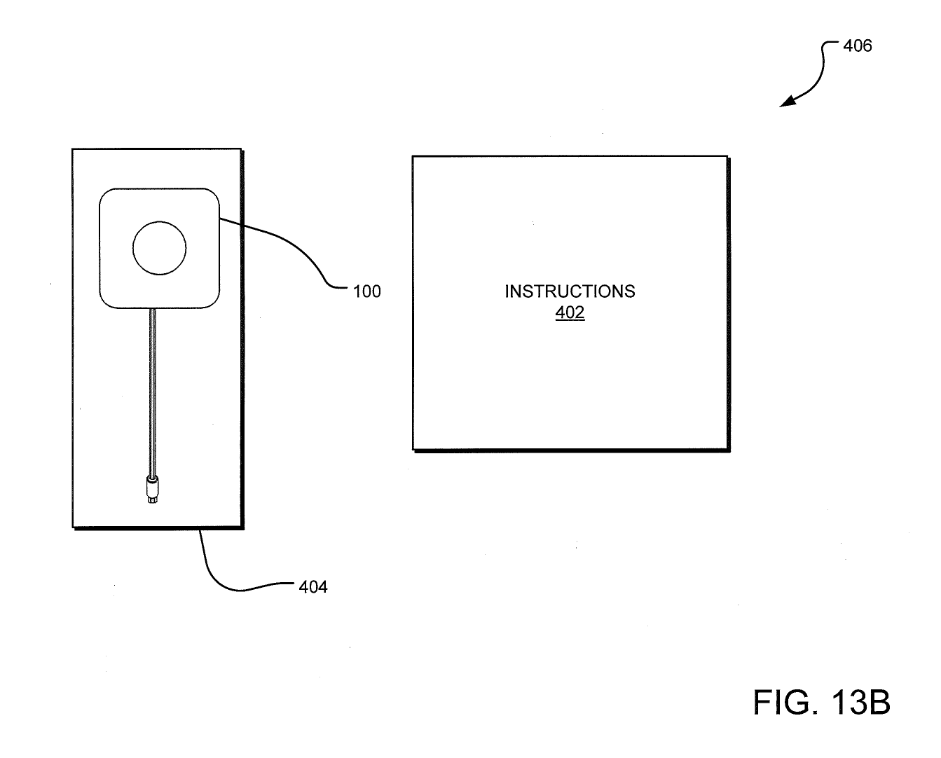

FIGS. 13A-13B show kits 400 and 406 including four patient interfaces 100 and one patient interface 100, respectively, and instructions 402. The patient interfaces 100 may be manufactured and sold as sterile pre-made kits 400, 406. In the implementation shown in FIG. 13A, the kit 400 includes four patient interfaces 100 applied to a peel-away membrane 404, which covers the patient side 104 of the anchor 102. The peel-away membrane 404 may be a cellophane, plastic, or other protective membrane fabric.

In another implementation shown in FIG. 13B, the kit 406 contains one patient interface 100 applied to the peel-away membrane 404. The kits 404, 406 may further include the instructions 402 on how to use the patient interfaces 100 with the probe 200, as described herein. In one implementation, the instructions 402 are folded paper. In another implementation, the instructions 402 are provided on the packaging. In still another implementation, the instructions 402 are provided via a communications network, such as the Internet. However, other means of providing the instructions 402 are contemplated. The kits 400, 406 may be sterilized and sealed for transportation and distribution.

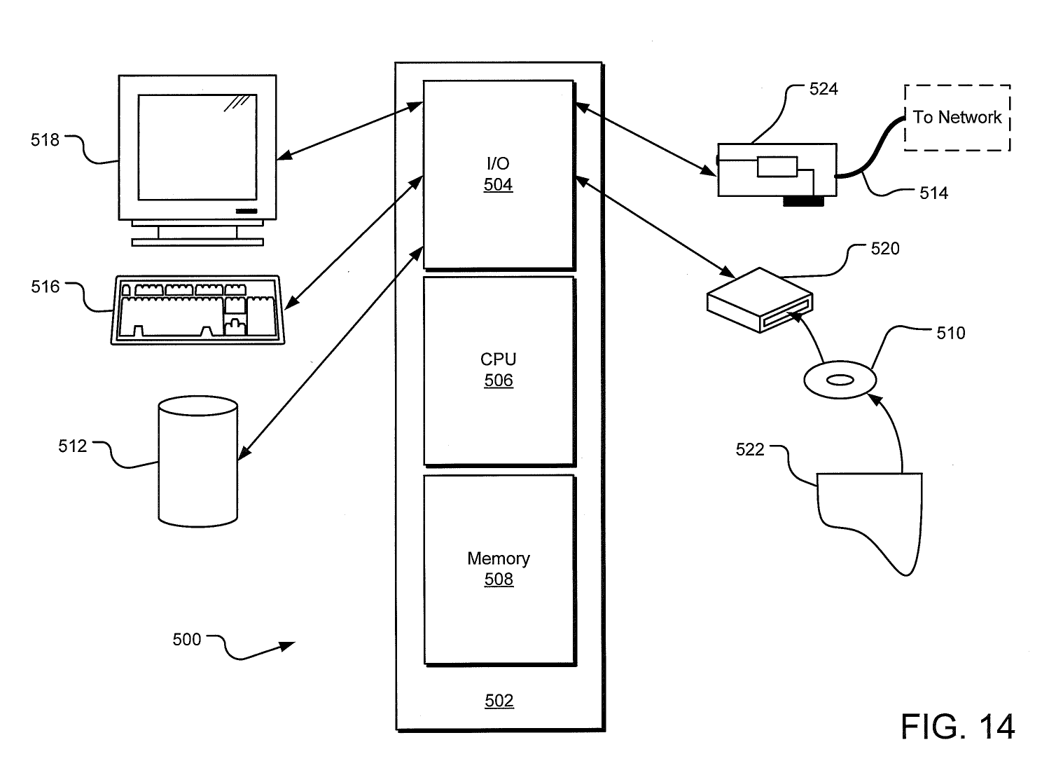

FIG. 14 illustrates an example computer system 500 that may be useful in implementing the presently disclosed technology. A general purpose computer system 500 is capable of executing a computer program product to execute a computer process. Data and program files may be input to the computer system 500, which reads the files and executes the programs therein. Some of the elements of a general purpose computer system 500 are shown in FIG. 5 wherein a processor 502 is shown having an input/output (I/O) section 504, a Central Processing Unit (CPU) 506, and a memory section 508. There may be one or more processors 502, such that the processor 502 of the computer system 500 comprises a single central-processing unit 506, or a plurality of processing units, commonly referred to as a parallel processing environment. The computer system 500 may be a conventional computer, a distributed computer, or any other type of computer, such as one or more external computers made available via a cloud computing architecture. The presently described technology is optionally implemented in software devices loaded in memory 508, stored on a configured DVD/CD-ROM 510 or storage unit 512, and/or communicated via a wired or wireless network link 514 on a carrier signal, thereby transforming the computer system 500 in FIG. 14 to a special purpose machine for implementing the described operations.

The I/O section 504 is connected to one or more user-interface devices (e.g., a keyboard 516 and a display unit 518), a disc storage unit 512, and a disc drive unit 520. Generally, the disc drive unit 520 is a DVD/CD-ROM drive unit capable of reading the DVD/CD-ROM medium 510, which typically contains programs and data 522. Computer program products containing mechanisms to effectuate the systems and methods in accordance with the presently described technology may reside in the memory section 504, on a disc storage unit 512, on the DVD/CD-ROM medium 510 of the computer system 500, or on external storage devices made available via a cloud computing architecture with such computer program products, including one or more database management products, web server products, application server products, and/or other additional software components. Alternatively, a disc drive unit 520 may be replaced or supplemented by a floppy drive unit, a tape drive unit, or other storage medium drive unit. The network adapter 524 is capable of connecting the computer system 500 to a network via the network link 514, through which the computer system can receive instructions and data embodied in a carrier wave. Examples of such systems include personal computers, Intel or PowerPC-based computing systems, AMD-based computing systems and other systems running a Windows-based, a UNIX-based, or other operating system. It should be understood that computing systems may also embody devices such as Personal Digital Assistants (PDAs), mobile phones, tablets or slates, multimedia consoles, gaming consoles, set top boxes, etc.

When used in a LAN-networking environment, the computer system 500 is connected (by wired connection or wirelessly) to a local network through the network interface or adapter 524, which is one type of communications device. When used in a WAN-networking environment, the computer system 500 typically includes a modem, a network adapter, or any other type of communications device for establishing communications over the wide area network. In a networked environment, program modules depicted relative to the computer system 500 or portions thereof, may be stored in a remote memory storage device. It is appreciated that the network connections shown are examples of communications devices for and other means of establishing a communications link between the computers may be used.

In an example implementation, one device 20 can be used or multiple devices 20 can be used to facilitate efficient acquisition of cardiac or other patient data points by placing the devices 20 at multiple vantage points on the patient 10. A user may access or otherwise interact with the cardiac or other patient data points using the computer system 500, which may include other computing devices, such as described herein. A plurality of internal and external databases, source databases, and/or data cache on the servers are stored as the memory 508 or other storage systems, such as the disk storage unit 512 or the DVD/CD-ROM medium 510, and/or other external storage devices made available and accessible via a cloud computing architecture. Algorithms, software, and other modules and services may be embodied by instructions stored on such storage systems and executed by the processor 502. Some or all of the operations described herein may be performed by the processor 502. Further, local computing systems, remote data sources and/or services, and other associated logic represent firmware, hardware, and/or software configured to perform some or all of the operations described herein. Such services may be implemented using a general purpose computer and specialized software (such as a server executing service software), a special purpose computing system and specialized software (such as a mobile device or network appliance executing service software), or other computing configurations. In addition, one or more functionalities of the systems and methods disclosed herein may be generated by the processor 502 and a user may interact with a Graphical User Interface (GUI) using one or more user-interface devices (e.g., the keyboard 516, the display unit 518, and the user devices 504) with some of the data in use directly coming from online sources and data stores. The system set forth in FIG. 14 is but one possible example of a computer system that may employ or be configured in accordance with aspects of the present disclosure.

The described disclosure may be provided as a computer program product, or software, that may include a machine-readable medium having stored thereon instructions, which may be used to program a computer system (or other electronic devices) to perform a process according to the present disclosure. A machine-readable medium includes any mechanism for storing information in a form (e.g., software, processing application) readable by a machine (e.g., a computer). The machine-readable medium may include, but is not limited to, magnetic storage medium (e.g., floppy diskette), optical storage medium (e.g., CD-ROM); magneto-optical storage medium, read only memory (ROM); random access memory (RAM); erasable programmable memory (e.g., EPROM and EEPROM); flash memory; or other types of medium suitable for storing electronic instructions.

The description above includes example systems, methods, techniques, instruction sequences, and/or computer program products that embody techniques of the present disclosure. However, it is understood that the described disclosure may be practiced without these specific details.

It is believed that the present disclosure and many of its attendant advantages will be understood by the foregoing description, and it will be apparent that various changes may be made in the form, construction and arrangement of the components without departing from the disclosed subject matter or without sacrificing all of its material advantages. The form described is merely explanatory, and it is the intention of the following claims to encompass and include such changes.

While the present disclosure has been described with reference to various embodiments, it will be understood that these embodiments are illustrative and that the scope of the disclosure is not limited to them. Many variations, modifications, additions, and improvements are possible. More generally, embodiments in accordance with the present disclosure have been described in the context of particular implementations. Functionality may be separated or combined in blocks differently in various embodiments of the disclosure or described with different terminology. These and other variations, modifications, additions, and improvements may fall within the scope of the disclosure as defined in the claims that follow.

What is claimed is:

1. A patient interface for securing a probe to a patient comprising:
an anchor having a patient side adapted for attaching to a surface of the patient and a probe side disposed opposite the patient side;
a synchronizer having a plurality of synchronization features corresponding to a plurality of imaging locations on the patient, the synchronizer mounted to the anchor and adapted to engage the probe; and
a window extending through the anchor and the synchronizer, the plurality of synchronization features positioned around the window, each of the synchronization features including a unique set of keys identifying one of the plurality of imaging locations.

2. The patient interface of claim 1, wherein the unique set of keys includes at least one of:
a unique number of keys; one or more uniquely shaped keys; one or more uniquely sized keys;
or one or more uniquely formatted keys.

3. The patient interface of claim 1, wherein the synchronization features are spaced evenly around the window.

4. The patient interface of claim 1, wherein each of the synchronization features is positioned at a right angle to an adjacent synchronization feature relative to a center of the window.

5. The patient interface of claim 1, wherein the plurality of imaging locations includes at least one of: a generic imaging location; a parasternal imaging location; an apical imaging location; or a subcostal imaging location.

6. The patient interface of claim 1, wherein at least one of the synchronization features has no keys in the unique set of keys.

7. The patient interface of claim 1, wherein each of the synchronization features includes a projection extending from a rim towards the window.

8. The patient interface of claim 7, wherein the projection includes a shelf formed by an indent in the projection, the unique set of keys being positioned on the shelf.

9. The patient interface of claim 7, wherein the projection include a channel adapted for engaging the probe.

10. The patient interface of claim 1, wherein the synchronizer is mounted to the anchor between the patient side and the probe side.

11. The patient interface of claim 1, wherein the patient side includes a membrane coated with an adhesive for attaching to the surface of the patient.

12. The patient interface of claim 1, wherein the anchor is a patch formed from one or more layers of biocompatible material.

13. The patient interface of claim 12, wherein the patch is four square inches in size.

14. The patient interface of claim 1, further comprising:
a recognizer having one or more processors configured to identify each of the plurality of imaging locations based on the unique set of keys of each of the synchronizing features.

15. The patient interface of claim 14, wherein the one or more processors are further configured to detect and authorize use of the probe.

16. The patient interface of claim 14, wherein the one or more processors are further configured to calibrate the probe.

17. The patient interface of claim 1, wherein the unique set of keys are configured to match a corresponding set of receptacles in the probe.

18. The patient interface of claim 17, wherein the match between the set of receptacles and the unique set of keys is at least one of: a mechanical match; an electrical match; a magnetic match; or an optical match.

19. The patient interface of claim 1, wherein the unique set of keys includes at least one contoured protrusion.

20. The patient interface of claim 1, wherein the plurality of imaging locations include locations for acquiring cardiac data points for hemodynamic management.

21. A probe comprising:
a housing having an interface side;
a head mounted within an opening in the interface side, the head configured to acquire data points from a patient; and
a synchronizer having a set of receptacles disposed in a groove extending around the opening, the set of receptacles configured to receive a set of unique keys from a plurality of sets based on an orientation of the set of receptacles to a patient interface, each of the plurality of sets corresponding to an imaging location on the patient.

22. A device comprising:
an anchor having a side adapted for attaching to a surface of a target;
a synchronizer having a plurality of synchronization features corresponding to a plurality of imaging locations on the target, the synchronizer mounted to the anchor;
a window extending through the anchor and the synchronizer, the plurality of synchronization features positioned around the window, each of the synchronization features including a unique set of keys identifying a particular imaging location from the plurality of imaging locations;
a probe housing having an interface side;
a head mounted within an opening in the interface side, the head configured to acquire data points from the target along an imaging direction;
a set of receptacles disposed adjacent to the opening in the interface side, the set of receptacles configured to match the unique set of keys in one of the synchronization features based on an orientation of the interface side to the window; and
a recognizer configured to identify the particular imaging location based on the match between the unique set of keys and the set of receptacles.

23. The device of claim 22, wherein the target is a patient.

24. The device of claim 22, wherein the match between the set of receptacles and the unique set of keys is at least one of: a mechanical match; an electrical match; a magnetic match; or an optical match.

25. A method for securing a probe to a patient interface, the method comprising:
attaching an anchor to a surface of a patient at a particular imaging location, the anchor mounted to a synchronizer having a plurality of synchronization features corresponding to a plurality of imaging locations on the patient, the plurality of synchronization features positioned around a window extending through the anchor and the synchronizer, one of the synchronization features including a unique set of keys identifying the particular imaging location from the plurality of imaging locations;
aligning a set of receptacles disposed adjacent to an opening in an interface side of a housing of the probe with the unique set of keys; and
coupling a body extending from the opening in the interface side to the synchronizer based on the alignment of the receptacles with the unique set of keys, the coupling positioning a head mounted within the opening in the interface side within the window, the head configured to acquire data points from the patient along an imaging direction at the particular imaging location.

26. One or more processor-readable storage media storing processor-executable instructions for performing a process, the process comprising:
identifying a match between a set of receptacles and a unique set of keys, the set of receptacles disposed adjacent to an opening in an interface side of a probe housing, the unique set of keys disposed on one of a plurality of synchronization features positioned around a window in a patient interface, the plurality of synchronization features corresponding to a plurality of imaging locations; and
determining a particular imaging location from the plurality of imaging locations based on the match between the set of receptacles and the unique set of keys.
